(19) United States
(12) Patent Application Publication
(10) Pub. No.: US 2022/0117525 A1
(43) Pub. Date: Apr. 21, 2022

(54) SENSOR AND SYSTEM FOR NEONATAL JAUNDICE MONITORING AND MANAGEMENT

(71) Applicant: NATIONAL UNIVERSITY OF SINGAPORE, Singapore (SG)

(72) Inventors: Dieter Wilhelm TRAU, Singapore (SG); Shihao LI, Singapore (SG)

(21) Appl. No.: 17/422,202

(22) PCT Filed: Jan. 13, 2020
(86) PCT No.: PCT/SG2020/050013

§ 371 Date: Jul. 12, 2021

(30) Foreign Application Priority Data
Jan. 11, 2019 (SG) 10201900285R

(51) Int. Cl. A61B 5/145; A61N 5/06; A61B 5/0205; A61B 5/1455; A61B 5/024

(52) U.S. Cl. CPC A61B 5/14546; A61N 5/0621; A61B 5/024; A61B 5/1455; A61N 2005/0663; A61B 5/02055

(57) ABSTRACT

The current invention is disclosing a sensor to monitor changes of bilirubin blood levels. The sensor of the current invention includes a bleaching light source(s) and a sensor light source(s) and a detector(s). The invention of the disclosed sensor lies in the bleaching light source in addition to a light source that is used for optical measurement. The bleaching light source is bleaching and photocatalytic, converting bilirubin into water soluble isomers/derivatives that are faster removed from the skin and secreted by the body. As a result, deposited bilirubin is removed in the light path of the sensor light source, and the non-invasive method of the present invention is measuring the bilirubin blood level without interference from deposited bilirubin in tissue, e.g. skin. The non-invasive optical sensor and method may be applied in monitoring the progression of jaundice and to manage jaundice phototherapy.

### TECHNICAL FIELD

The present disclosure relates to a non-invasive optical monitoring sensor for jaundice and a system to manage jaundice. Advantageously the non-invasive method of the present invention is measuring, the bilirubin blood level without interference from deposited bilirubin in tissue, e.g. skin. The non-invasive optical sensor and method may be applied in monitoring the progression of jaundice and to control jaundice phototherapy.

### BACKGROUND

Jaundice is a medical condition caused by the insufficient ability of the liver to remove bilirubin from the body. Accumulation of bilirubin leads to yellow discoloration of the skin, the gums, and the sclera of a subject. Left untreated, the medical dysfunctions causing jaundice may be fatal in adults and/or may cause brain damage in neonates. Jaundice screening of a subject such as in an adult or infants during neonatal care may be performed by invasive blood tests to detect bilirubin levels in the blood of the subject.

Jaundice may also be detected by noninvasive methods by analyzing the skin color of a subject by visual means or by using an apparatus. Analysis by visual means is qualitative and subjected to the skills of the health professional. Analysis using an apparatus can be performed by reflection photometry or imaging. Transcutaneous jaundice meters typically use reflection photometry and used typically on the forehead or sternum to measure bilirubin levels.

The problem with all non-invasive approaches is that analyzing or imaging the yellow color of the skin is indicating jaundice but is not directly reflecting the bilirubin blood level and the changes in the bilirubin blood level.

Thus, there is a need for an accurate non-invasive method that is directly reflecting the serum bilirubin level without interference of pigmentation or bilirubin already deposited within the skin or tissue of a subject.

### SUMMARY

The current disclosure provides a solution for the inability of current transcutaneous jaundice meters to measure bilirubin blood levels. Instead current transcutaneous jaundice meters measure the level of deposited bilirubin in the skin or tissue.

There is thus provided, in accordance with some embodiments of the present disclosure, a solution of the problem stated above and a non-invasive optical sensor and system is introduced to measure bilirubin blood levels. Advantageously, such measurements are not affected by deposited bilirubin in the tissue, e.g. skin, and provide more accurate measurement of bilirubin blood levels and its changes.

The current invention is disclosing a sensor to monitor changes of bilirubin blood levels. The sensor of the current invention includes a bleaching light source(s) and a sensor light source(s) and a detector(s). The invention of the disclosed sensor lies in the bleaching light source in addition to a light source that is used for optical measurement. The bleaching light source is bleaching and photocatalytic, converting bilirubin into water soluble isomers/derivatives that are faster removed from the skin and secreted by the body. As a result, deposited bilirubin is removed in the light path of the sensor light source and thus the result is not influenced by deposited bilirubin and instead represents the serum bilirubin. For better understanding of the current disclosure, the following definitions are made.

### DEFINITIONS

in the context of the present invention, the term “stationary bilirubin” shall mean bilirubin deposited in the tissue, e.g. skin.

In the context of the present invention, the term “mobile bilirubin” shall mean bilirubin within the blood and transported by the blood flow in tissues, e.g. skin. The mobile bilirubin represents serum bilirubin and its level is similar to the serum bilirubin level.

In the context of the present invention, the “bleaching light source” has the function to emit light to bleach and photocatalytic convert bilirubin into water soluble derivatives that are faster removed from the skin and secreted by the body. The wavelength of the bleaching light source is generally where bilirubin is most efficient removed from the body. In particular, this wavelength is in the range from 400 to 600, which covers the absorption peak of bilirubin at 450 nm. It is preferred that the light output is strong to remove all stationary bilirubin in the light path of the bleaching light source. The bleaching light source may be a light-emitting diode (LED) or laser diode emitting continuous or pulsed irradiation.

In the context of the present invention, the “sensor light source” is emitting light at the s absorbance wavelength of bilirubin. Typically, this wavelength is in the range of 400 nm to 500 inn and most preferred at the absorbance maxima of bilirubin at 450 nm. The sensor light source light path is in the light path of the bleaching light source. Thereby, absorbance at the sensor light source wavelength is caused by mobile bilirubin because the stationary bilirubin was removed by the action of the bleaching light source.

In the context of the present invention, the “detector” is a light detector, e.g. a photodiode, CCD or CMOS sensors that generate electrical signals such as currents or potentials proportional to the light received from the sensor light source and the bleaching light source. The sensitivity of the detector is in the wavelength range of 400 inn to 600 nm. More than one detector may be used with different sensitivity range and response range. The signals of the detector or multiple detectors is in the sensing device reflect the bilirubin deposited in the tissue and/or bilirubin present in the blood.

In the context of the present invention, “processors or microprocessors” are computers or microcontroller units, that can provide electrical outputs computed from inputs received from a detector, such as a light detector. The electrical output can be an output to display units and/or alerting units such like speakers/beepers or an output remotely transmitted to other devices or stored on a data media.

In the context of the present invention, the “normalization light source” is operated at a wavelength at which bilirubin is not absorbing. It is used to normalize the signal(s) of the sensor light source. Thereby the normalization light source and its corresponding detector is not influenced by bilirubin levels or its changes.

### BRIEF DESCRIPTION OF THE DRAWINGS

Some non-limiting exemplary embodiments or features of the disclosed subject matter are illustrated in the following drawings.

FIG. 1A illustrates the optical sensor in contact with the skin for jaundice monitoring composed of a bleaching light source operated when the sensor light source is off, and a sensor light source operated when the bleaching light source is off, and the detector shall operate when the sensor light source is on and wherein such detector senses a signal proportional to mobile bilirubin. There could be multiple light sources and detectors as shown in FIG. 1B.

FIG. 1C illustrates the bleaching light source and measurement light source combined together.

FIG. 1D illustrates different setups for bilirubin measurement with the inventive bleaching light source and without the bleaching light source.

FIG. 2A illustrates the reflection mode of the sensor, in where the light sources and the detector are placed on the same side. Sensors could measure bilirubin level after the transcutaneous bilirubin is bleached. And could also measure the signal from a bleached area and a non-bleached area like in FIG. 2B.

FIG. 3 schematic illustrating a system to manage jaundice composed of the sensor, a control box and a light module.

FIG. 4 illustrates the workflow of the sensor and control system to measure bilirubin and manage jaundice.

With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

Identical or duplicate or equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

### DETAILED DESCRIPTION

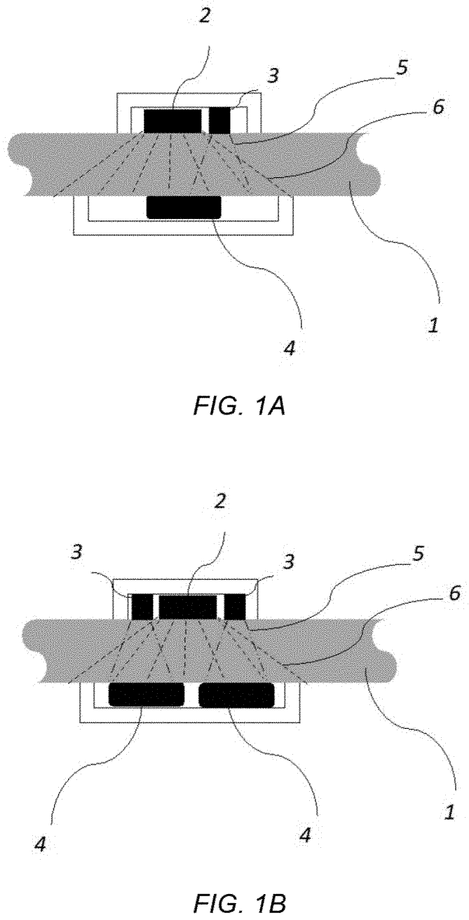

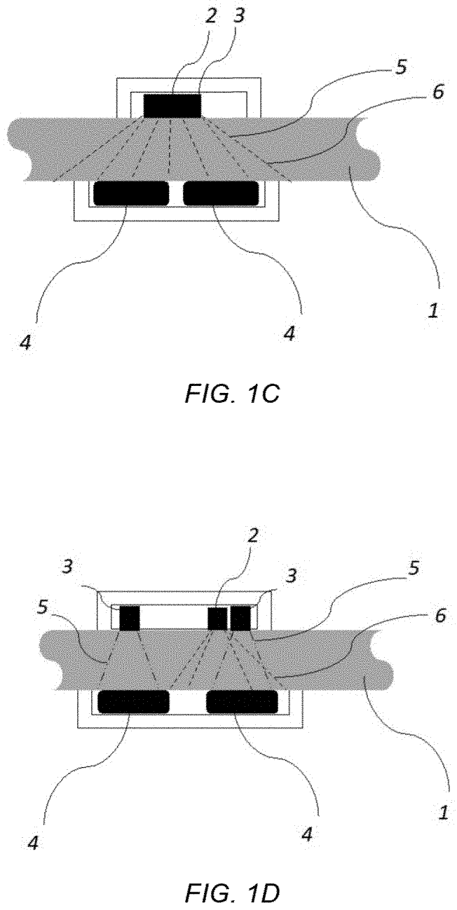

The apparatus and Method provided herein according to some embodiments of the present disclosure, enable the non-invasive determination of bilirubin levels in blood by detecting bilirubin from a region of tissue, e.g. skin, which is firstly illuminated by the bleaching light source for the removal of the transcutaneous bilirubin, herein also called the stationary bilirubin. As illustrated in FIG. 1A, the light path of the sensor light source 5 lies within the light path of the bleaching light source 6. Advantageously, the sensor of the present invention is not influenced by deposited bilirubin and provides a signal proportional to the serum bilirubin. When the bleaching light source lo 2 is switched on, the bleaching light source 2 is emitting light causing the bleaching and removal of stationary bilirubin from tissue, e.g. skin 1 in the light path of the sensor light source 5. The bleaching light source may be operated in a continuous or pulsed fashion taking into consideration photoisomerization parameters like absorption and extinction coefficients, quantum yield, radiative lifetime, energy-gap calculation, and zero-and-first order kinetics. The emission duration and frequency of the bleaching light source will be dependent on the continuous or pulsed configuration of the bleaching light source and the photoisomerization parameters. Thereby, stationary bilirubin is removed or the rate of removal of stationary bilirubin is determined through an iterative and integration-based algorithm of numerous datapoints collected over a period of time comparatively matched with serum bilirubin levels taken through a single class or multiple class of detectors. When the bleaching light source 2 is switched off and the sensor light source 3 is switched on, bilirubin present in the light path of the sensor light source is mobile bilirubin and the signal received by the detector 4 corresponds to the serum bilirubin only. Because the penetration depth of blue light is only about 1 to 2 mm into the skin, the sensor of FIG. 1 can have two bleaching light sources opposite of each other. If the sensor of FIG. 1 is attached onto the ear, specifically onto the earlap a bleaching light source is present on both sites of the earlap. This configuration reaches a deeper penetration into the tissue because the tissue of the earlap is illuminated from both sited. The combined penetration depths of up to 4 mm is larger than the thickness of an infant earlap and consequently all stationary bilirubin can be bleached within the earlap of an infant. This approach is not limited to the ear but can be applied to the finger or toe or any other tissue.

This is an advantage over current non-invasive bilirubinometers, whose signals are always the sum of the stationary and mobile bilirubin. Or in most cases wherein the signal only reflects the stationary bilirubin, adipose tissue and other pigmentations. The effect of the bleaching light source on the blood bilirubin levels is negligible. Even some blood or mobile bilirubin bleached at the light pass of the bleaching light source is replaced within a short time interval by the blood flow through the tissue. The present invention is an advancement in the field enabling the measurement of blood bilirubin changes by the introduction of a novel bleaching light source within a bilirubin sensor.

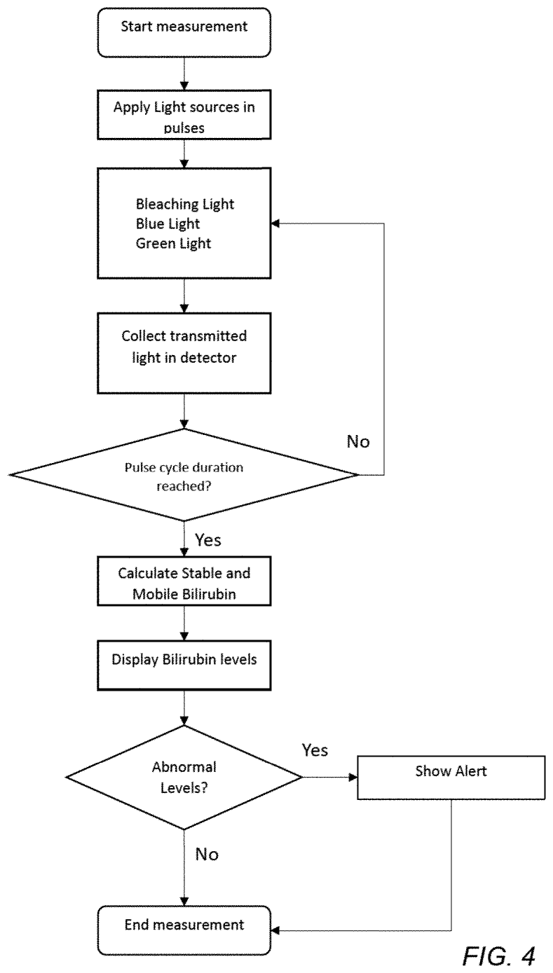

When in operation as shown in FIG. 4, the sensor after receiving an appropriate activation command from the processor will send a signal to the bleaching light source 2 to generate bleaching light in a wavelength (ex. 450 nm). The bleaching light may be transmitting light most lo of the time the device is in contact with skin or tissue to remove as much as possible of stationery bilirubin. The emitted light passes through skin 1. Light transmitted through 1 may be collected by detector 4. Then the pulse cycle duration will be assessed by the control box before the calculation of the bilirubin level, then the results will be displayed, as the workflow in FIG. 4. The resulting signal may be sampled by the control box. This procedure will be done in continuous or pulsed fashion with the resulting signal being recorded over a period of time.

Once the integration process is completed, another signal is sent from the processor to light source 3 to emit light radiation in a second wavelength (ex. 520 nm) and after a delay another in a third wavelength (ex. 470 nm, for example). The emitted light travels the same path as the bleaching light to the detectors, which measures the magnitude of light transmitted through the tissue or skin or through an earlap. The resulting signal may be recorded.

For the calculation of bilirubin levels, first the isomerization of bilirubin levels will be measured through the integration of the signals. Bilirubin is photoisomerized to 4Z, 15E bilirubin instantaneously upon exposure to photocatalytic light. The light pulses consisting of bleaching light, followed by blue and green wavelength light. By using the methods of femtosecond transient absorption spectrometry with anisotropy measurements through appropriate filters, the photoisomeration of bilirubin to water soluble 4Z, 15E bilirubin will be calculated.

Similarly, the resulting light after blue and green wavelength light irradiation when measured by the detector will give a combined measurement of stationary and mobile bilirubin. As the fraction of mobile bilirubin will remain more or less constant, the rate of bilirubin to 4Z, 15E bilirubin will be that of the stationary bilirubin component. Since the decay of bilirubin to 4Z, 15E bilirubin is follows first-order kinetics, levels of stationary bilirubin can be calculated. Then, through these techniques, the pulsed measurements, when integrated over time, will provide the levels of stationary bilirubin concentration and mobile bilirubin concentration. These calculations will be done in the processor.

In another embodiment of the sensor depicted in FIG. 1B, multiple light sources 2, 3 and detectors 4 are presented in this embodiment. The light source 2 is the bleaching light source and s includes a reference light source 3 with a different wavelength that does not lie in the absorption/fluorescent spectrum range of bilirubin.

Meanwhile, multiple detectors 4 shall be used in this embodiment. The detectors will be equipped with different response range and sensitivities, for the purpose of detecting transmittance, absorption and fluorescent signals, which include bleaching signal, sensing signal and reference signal.

When the bleaching light source 2 is switched on, the bleaching light source 2 is emitting light causing the bleaching and removal of stationary bilirubin from tissue, e.g. skin 1 in the light path of the sensor light source 5. Thereby, stationary bilirubin is removed or the rate of removal of stationary bilirubin is determined. When the bleaching light source 2 is switched off and the sensor light source 3 is switched on, bilirubin present in the light path of the sensor light source is mobile bilirubin and the signal received by the detector 4 is corresponding to the serum bilirubin concentration only. Because the penetration depth of blue light is only about 1 to 2 mm into the skin, the sensor of FIG. 1B can have two bleaching light sources and two sensor light sources opposite of each other.

In FIG. 1C, another embodiment of the sensor is presented, the bleaching light source 2 and sensing light source 3 are combined together into one. In this embodiment, the bleaching light source 2 will be initiated first to breakdown and clear bilirubin that deposited in the tissue. Then the power and frequency will adjust to the sensing light source to start the measurement of serum bilirubin in the blood. The combined light sources 2 and 3 will work alternatively to remove the bilirubin in the tissue 1 and measure serum bilirubin in the blood. The sensor(s) 4 will detect the signals from bleaching light source 2 and sensing light source 3, which in this case are combined into one light source.

In FIG. 1D, another embodiment of the sensor 4 is presented, the sensor contains an additional sensor light source 2 and sensor 4 wherein this additional sensor light sources' light path 5 is not within the area of the tissue, e.g. skin 1 bleached by the bleaching light source 2. With this double sensor configuration, the difference of the stationary bilirubin is measured for an area of tissue illuminated with bleaching light and an area of tissue not illuminated with bleaching light. This configuration also provides an indicator of the ability of the body to remove bilirubin through a differential calibrated measurement providing, information of the kinetics of stable and mobile

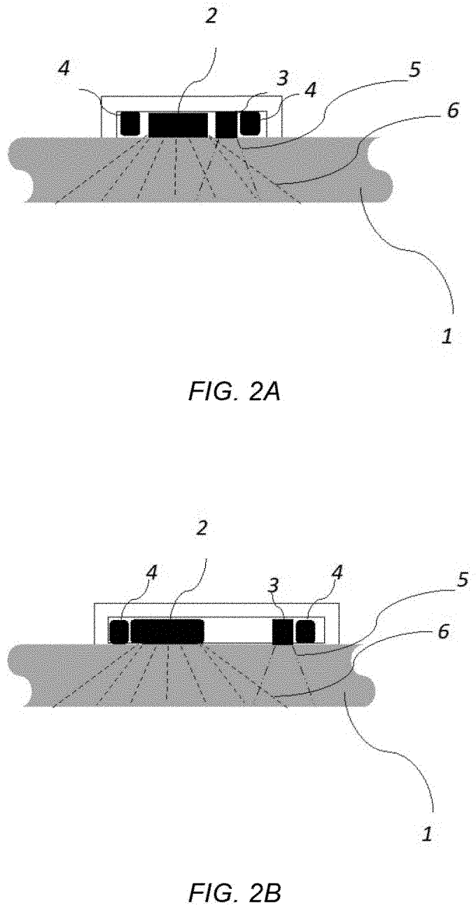

In FIG. 2A, one more embodiment of the sensor is presented, in which the detector(s) 4 and light source(s) 2 are placed on the same side of the tissue, e.g. skin 1. The bleaching light source 2 will be initiated first to remove the bilirubin in the tissue, e.g. skin 1, then the sensing light source 3 ardor reference light source 3 will be on sequentially, to detect serum bilirubin. Detectors 4 will detect the signals from the same side of the tissue, e.g. skin 1.

In FIG. 2B, the sensor contains an additional sensor light source 3 and sensor 4 wherein this additional sensor light sources light path 5 is not within the area of the tissue, e.g. skin 1 bleached by the bleaching light source 2. With this double sensor 4 configuration placed at the same side with the light sources 2, 3 the difference of the stationary bilirubin is measured. This configuration also provides an indicator of the ability of the body to remove bilirubin.

The changes in the concentration for stationary bilirubin and mobile bilirubin is recorded over time. Changes in stationary bilirubin may be slow due to the relative slow kinetics of the removal of the bilirubin from the skin. Therefore, measurements of the stationary bilirubin provided by state of the art non-invasive devices are not reflecting the changes in blood bilirubin levels. The present invention overcomes this limitation by the introduction of a bleaching light source.

The system and method of the current invention solve the problem of non-invasive neonatal or children jaundice management by determining the concentration of bilirubin levels in the blood of the subject.

In the above embodiments of the invention a sensor contact with tissue, e.g. skin of a subject is disclosed.

The embodiments taught herein overcome the problem that deposited bilirubin in the tissue, e.g. skin, causes an error in the determination of bilirubin resulting in either an overestimation or underestimation of bilirubin concentration levels in the blood of the subject.

In another embodiment of the current invention the sensor in contact with the skin has two sensor light sources as disclosed in FIG. 2B. One light source is within the light pass of a bleaching light source and the other sensor light source is not. With this setup, the sensor light source and its respective detector within the light pass of a bleaching light source is measuring mobile bilirubin alone and the sensor light source and its detector not in the bleaching light pass is measuring the sum of the mobile and stationary bilirubin. By subtracting the signals of the two light sources received by their respective detectors the amount of stationary bilirubin can be calculated.

In general, the Wading light source May be switched on most of the time to remove stationary bilirubin. Alternative the bleaching light source can be pulsed or only switched on for certain periods. For measurements, the bleaching light source can be switched off and the measurement light source is switched on. Measurements can be performed at any regular or non-regular time interval or if requested by a user. The measurement time is in general very short, between microseconds and seconds. More than one measurement may be performed, and results may be processed such as the median is calculated or statistical analysis is applied to the results. It is possible to perform a measurement when the bleaching light source is on. However, this is not preferred because the bleaching light may reduce the accuracy or saturate the detector. The detector may be used also to measure the intensity of the bleaching light source.

The sensor of the current invention may contain a proximity sensor. The proximity sensor may be used to monitor the attachment of the sensor to the skin of a subject and to provide warnings.

The sensor of the current invention may contain a temperature sensor. The temperature sensor may be used to monitor the correct attachment of the device and the health of the subject and to provide warnings.

The sensor of the current invention may contain a pressure sensor. The pressure sensor may be used to monitor the heartbeat and to correct signals from the light detection sensors and to monitor the health of the subject and to provide warnings.

The combined signals of the temperature sensor and the pressure sensor may be used to confirm that the device is correctly attached to the subject.

The sensor of the current invention can be contacted with the skin by multiple means, such as by pressure, or by a clip mechanism or a bandage or a strap or by fixation onto the skin with an adhesive or mixtures thereof.

In general, the sensor of the present invention can be contacted to the skin at any part of the body. Preferentially the sensor is attached to the ear, or a finger or toe.

The sensor of the current invention can be contacted with the skin of a subject for any duration of time from minutes to weeks. If used on infants, the sensor may be brought into contact with the skin at the first or second day after birth. At this early time there is no bilirubin deposited in the skin or tissue of a newborn. Advantageously, the bleaching light source of the sensor will prevent deposition of bilirubin in the light pass of the sensor light source and thereby allows the measurement of the Mobile or Wed bilirubin and its changes alone. If the sensor of the current invention is brought into contact with the skin at the first or second day after birth, the bleaching light source will prevent any deposition of bilirubin at the light pass of the sensor light source and thereby any Change detected by the detector receiving light from the sensor light source is directly proportional to changes in the blood bilirubin levels measured in real time. By using a two-point calibration disclosed in the following, the relative change of the bilirubin measured as disclosed in the paragraph above can be linked to an absolute bilirubin level in the blood of the subject. First, the sensor of the current invention is attached to a newborn subject at the first or second day of birth and the detector signal is measured. The blood bilirubin is measured by state of the art is invasive methods and the two values are linked. Second, the blood bilirubin is measured at any following day and the sensor signal is measured at the same time and the results are linked. The differences of the second sensor signal to the first sensor signal and the corresponding differences of the second bilirubin blood level to the first bilirubin blood level provide an absolute calibration of the sensor with the unit of sensor signal per blood bilirubin concentration change. After the calibration is performed not only the relative changes are measurable but also the absolute changes of the bilirubin levels in the blood of a subject are measurable.

In another embodiment of the current invention a light source for normalization or comparison is added. The normalization light source is operated at a wavelength at which bilirubin is not absorbing light. Thereby the normalization light source and its corresponding detector is not influenced by bilirubin levels or its changes. The normalization light source and the corresponding detector signal shall be constant. However, small changes in the position of the sensor attached to the skin of the subject may change the light pass and thereby sensor outputs for the sensor light source. By using the signals from the corresponding detector of the normalization light source such changes can be corrected.

The embodiments taught herein are not united to infants and children but may include adults and/or animals (e.g. any living subject).

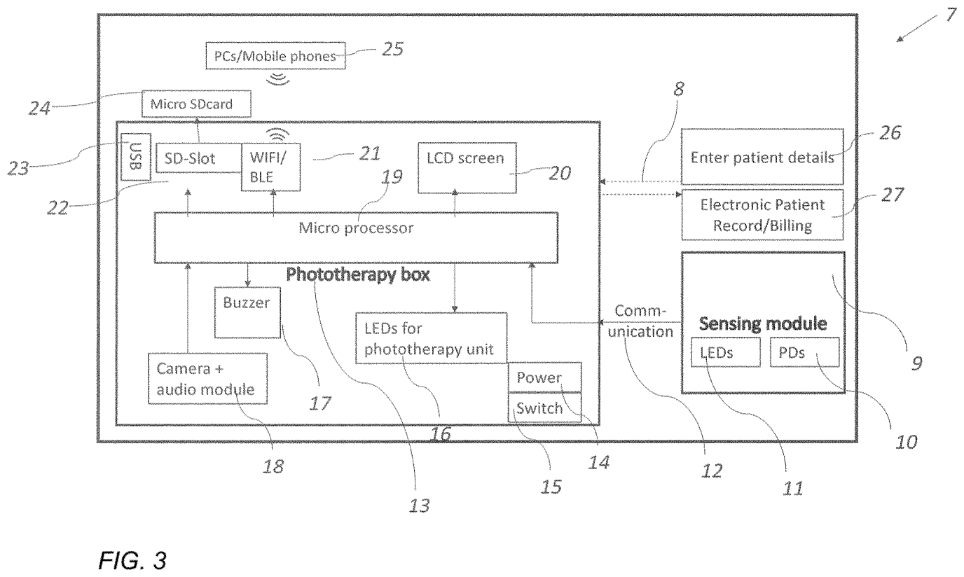

In a next embodiment of the current invention a system for the management of jaundice is disclosed, as shown in FIG. 3. The system consists of a sensor of the current invention in contact with the skin for jaundice monitoring composed of A bleaching light source operated when the sensor light source is off, and a sensor light source operated when the bleaching light source is off and a detector operated when the sensor light source is on and wherein such detector senses a signal proportional to mobile bilirubin. A control box to receive the signal from the sensor and to compute an output control signal. An illumination module to receive the output control signal and in respect to the output control signal controls the illumination power between 0 and 100% over time. FIG. 3 schematically illustrates a system to manage jaundice, which includes a sensing module 9 and a phototherapy box 13. Within the sensing module 9, photodiodes 10 and LEDs 11 are used to monitor stationary bilirubin level and mobile bilirubin level. The sensing module 9 will communicate with phototherapy box 13 with a wired/wireless communication method 12. The concentration may be displayed on a screen 20, or monitor 20, or display on a remote communication device 25. The bilirubin concentration or its change may be displayed in any suitable units, but typically in mg/dL. On the phototherapy box 13, there are power 14 and switch 15 that could initiate the sensing module 9 and phototherapy box 13. LEDs 16 are used for the treatment purpose. The intensity of the illumination module or LEDs 16 is controlled by the microprocessor 19, based on the signals sent through a wired/wireless communication 12 from sensing module 9. The Intensity of the light illumination to treat an infant is controlled between 0 and 100% over time. When there is any regulation of the intensity of the LEDs 16, the buzzer 17 will notify nurses/doctors with alerting sounds and/or notifications through WIFI/BLE 21. In this embodiment of the present disclosure, the data may be stored in memory card 24, which shall bel placed at the SD-slot 22, after being processed. In some cases, camera and audio module 18 may be used to record the video and audio of a subject in a phototherapy box, the data will be used to analyse the status of a subject during treatment. The data mentioned above from sensing module 9 and phototherapy box 13 may permit sharing between doctor's offices and wards, through USB 23 or WIFI/BLE 21. The jaundice management system in this embodiment shall facilitate patient data recording/management 26 and billing 27. The sensor in contact with the skin may be placed at any location of the body of a subject. A preferred location is on the ear. Another preferred location is on forehead or a finger of a subject.

The light sources for the sensor light source or the bleaching light source may be selected from xenon flash lamp or certain wavelengths light emitting diodes, a laser diode, and a polychromatic light source. A bandpass filter may be added in front of the light source to further select the emitted light wavelength.

The sensing module will provide the bilirubin concentration or the change of the bilirubin in the body, thus, provide guidance or instructions to the phototherapy device which could regulate s the intensity of the light treatment to a subject, the and inform caregivers by a buzzer when there is any change of the intensity. A camera and audio module may be used in certain cases, e.g. homecare, which could connect with PC/mobile to give continuous monitoring of a subject in the phototherapy box. The audio and video data may be analysed by artificial intelligence to tell the comfortless of a subject during treatment.

In some embodiments of the present disclosure, a method of reflection photometry is used. Incident light from the sensor light source may be directed onto the tissue. The reflected light may be collected in the at least one detector and the intensity of the reflected light may be measured. In the presence of bilirubin, the intensity may be reduced. With the increase of the bilirubin concentration in the blood, the absorption of 400 nm to 500 nm will increase resulting in a decrease in the intensity of reflected light.

In some embodiments of the present disclosure, the method of fluorescence emission by bilirubin may be used to determine the bilirubin concentration in the blood of the subject. In this case, incident light from the sensor light source may be used to excite bilirubin molecules. The emitted fluorescent light may be collected by the detector and the intensity measured. In the presence of bilirubin, the measured intensity increases.

The detector may include fluorescence detector such as a photodiode, a spectrometer, or camera. The received light by the detector, for example a photodiode or a spectrometer, may include fluorescence emission from the mobile bilirubin. A filter may be installed in front of the detector to select certain wavelength, e.g. the peak fluorescent emission wavelength of bilirubin, that can reach the detector and block out any other wavelength, e.g. the excitation wavelength.

In some embodiments of the present disclosure, the detector may be selected from the group consisting of a photodiode, a photomultiplier tube, a photoresistor, a charge coupled device (CCD) sensor, a complementary metal-oxide semiconductor (CMOS) sensor, a fluorescence detector, a filtered photodiode, a spectrometer, and a camera.

1. A sensor in contact with the skin for jaundice monitoring composed of:
A) a bleaching light source operated when the sensor light source is off;
B) a sensor light source operated when the bleaching light source is off;
C) a detector operated when the sensor light source is on;
wherein, if switched on the bleaching light source is emitting light causing the bleaching and removal of stationary bilirubin from the skin in the light path of the sensor light source and when the sensor light source is on, light received by the detector was transmitted through mobile bilirubin but not through stationary bilirubin resulting in a sensor signal proportional to mobile bilirubin and thereby measuring blood bilirubin levels.

2. The sensor of claim 1, wherein the bleaching light source is in the wavelength range of 400 nm to 600 nm.

3. The sensor of claim 1, wherein the sensor light source is in the wavelength range of 400 600 nm and most preferable at the absorbance maxima of bilirubin at 450 nm+/−20 nm.

4. The sensor of claim 1, wherein the detector detects light of the emission wavelength of the sensor light source or the demission wavelength of bilirubin.

5. The sensor of claim 1, wherein the light emitted by the sensor light source and received by the detector is transmitted through the skin or tissue or reflected by the skin or tissue or scattered by the skin or tissue or a mixture e thereof.

6. The sensor of claim 1, wherein the light emitted by the sensor light source is transmitted through the tissue of a subject and absorbed by bilirubin before it is received by the detector.

7. The sensor of claim 1, wherein the light emitted by the sensor light source is reflected and or scattered by the tissue of a subject and absorbed by bilirubin before it is received by the detector.

8. The sensor of claim 1, wherein the light emitted by the sensor light source is exciting bilirubin molecules and the emitted light from bilirubin molecules is received by the detector.

9. The sensor of claim 1, wherein the bleaching light source and the sensor light source is the same light source.

10. The sensor of claim 1, wherein the normalization light source and the signal light source and the bleaching light source are operated continuously, or they are pulsed.

11. The sensor of claim 1, wherein the sensor contains a sural light source not in the light pass of the bleaching light source.

12. The sensor of claim 1, wherein the sensor contains a normalization light source and a respective detector to normalize signals of a signal light source and its respective detector.

13. The sensor of claim 1, wherein the sensor contains a temperature and/or a pressure sensor to determine body temperature and/or heart beat.

14. A system for the management of jaundice composed of:
A) a sensor in contact with the skin for jaundice monitoring composed of A bleaching light source operated when the sensor light source is off, and a sensor light source operated when the bleaching light source is off and a detector operated when the sensor light source is on and wherein such detector senses a signal proportional to mobile bilirubin;
B) a control box to receive the signal from the sensor and to compute an output control signal;
C) an illumination module to receive the output control signal and in respect to the output control signal controls the illumination power between 0 and 100% over time.

15. The system of claim 14, wherein the signal from the sensor is directly controlling the light output power of the illumination module without the control box.
